(19) United States
(12) Patent Application Publication
(10) Pub. No.: US 2022/0395178 A1
(43) Pub. Date: Dec. 15, 2022

(54) Optical Intraocular Pressure Sensor in Cornea for Free-Space Interrogation

(71) Applicant: Twenty Twenty Therapeutics LLC, South San Francisco, CA (US)

(72) Inventors: Supriyo Sinha, Menlo Park, CA (US); Oleg Rumyantsev, Menlo Park, CA (US); Jeremy Chan, San Jose, CA (US); Dimitri T. Azar, Chicago, IL (US)

(73) Assignee: Twenty Twenty Therapeutics LLC, South San Francisco, CA (US)

(21) Appl. No.: 17/827,034

(22) Filed: May 27, 2022

Related U.S. Application Data
(60) Provisional application No. 63/209,277, filed on Jun. 10, 2021.

(51) Int. Cl. A61B 3/16; A61B 3/15; A61B 3/00

(52) U.S. Cl. CPC A61B 3/16; A61B 3/152; A61B 3/0025

(57) ABSTRACT

An intraocular pressure (IOP) measurement system. An optical pressure sensor is implantable in the cornea of an eye, wherein the sensor has a sealed cavity that changes shape as a function of IOP of the eye. An optical transmitter that is outside of the eye emits an incident optical beam. A receiver that is also outside of the eye produces an output signal in response to receiving reflections of the incident beam from the sensor. A processor is configured to estimate the IOP of the eye based on processing the output signal of the receiver. Other aspects are also described and claimed.

This patent application claims the benefit of the earlier filing date of U.S. provisional application No. 63/209,277 filed Jun. 10, 2021.

### FIELD

The subject matter of this disclosure relates to techniques for measuring intraocular pressure in human eyes. Such techniques are useful for treating and/or monitoring progression of eye diseases including glaucoma, but are not limited to use with the treatment of eye disease.

### BACKGROUND

Intraocular pressure (IOP) refers to the pressure of a fluid referred to as the aqueous humor inside the eye. The pressure is normally regulated by changes in the volume of the aqueous humor, but some individuals suffer from disorders, such as glaucoma, that cause chronic heightened IOP. Over time, heightened IOP can cause damage to the eye's optical nerve, leading to loss of vision. Presently, treatment of glaucoma mainly involves periodically administering pharmaceutical agents to the eye to decrease IOP. These drugs can be delivered, for example, by injection or eye drops. However, effective treatment of glaucoma requires adherence to dosage schedules and a knowledge of the patient's IOP. The more current or recent the measurement is, the more relevant it will be and hence the more effective the resulting treatment can be. The IOP for a given patient can vary significantly based on time of day, exercise, how recently a medication was taken, and other factors. Typically, IOP measurements are performed in a doctor's office and often no more than once or twice per year. These infrequent measurements are less able to account for variation in the patient's IOP, and may become stale due to the length of time between them. This means that any given measurement is subject to uncertainty, so it may take several IOP measurements over time to have confidence in the health of the patient's eye.

Typically, the IOP is measured using a tonometer, which is a device that is outside the eye and thus does not require a sensor within the eye. Contact tonometry is performed in a clinical setting, and the procedure requires numbing of the patient's eye, resulting in both inconvenience and discomfort. Noncontact tonometry involves directing a puff or jet of air towards the patient's eye and measuring the resulting deflection dynamics of the cornea. However, this requires a bulky and power hungry pump arrangement that may not be practical for home use, and is not as accurate as contact tonometry.

### SUMMARY

A minimally invasive, passive optical sensor is implanted in the cornea of a person's eye, and is used to measure the IOP of the eye. To do so, a reader (an electronic device that may be portable, battery powered, and held by the person themselves) is aligned with the eye, and an optical beam is emitted by a transmitter inside the reader. The reader is not physically attached to the sensor and may be outside of the eye. The beam may travel through free space (the ambient environment outside of the eye) and then enters the cornea where it impinges upon the sensor and is reflected by the sensor, towards a receiver in the reader. The reflection changes as it follows the changing IOP of the eye (for example over the course of a day). The sensor is passive in that it does not have a source of stored power that is used to transmit a signal containing information about the IOP. Instead, a part of the sensor that is reflecting the incident beam will bend or compress, as a function of the nearby IOP, resulting in the reflection changing accordingly. An estimate of the IOP is then determined by digitally processing an electrical output signal of the receiver (that is responsive to the reflections that travelled from the sensor and then through the ambient environment before impinging on the receiver.)

As the sensor is passive, it can be made small and thin so as to be implanted into the cornea in a minimally invasive manner, more easily and with less risk of complications as compared to implant locations that are further inside the eye. In one aspect, the sensor and the reader together are part of a consumer-focused solution that enables more frequent IOP measurements to be made by the patient at home, which are important for monitoring the progression of glaucoma and the effectiveness of any treatments.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings, in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 shows an example of how to measure IOP using a passive, implanted optical pressure sensor.

FIG. 2 illustrates a front view of the eye whose cornea contains a passive implanted optical pressure sensor.

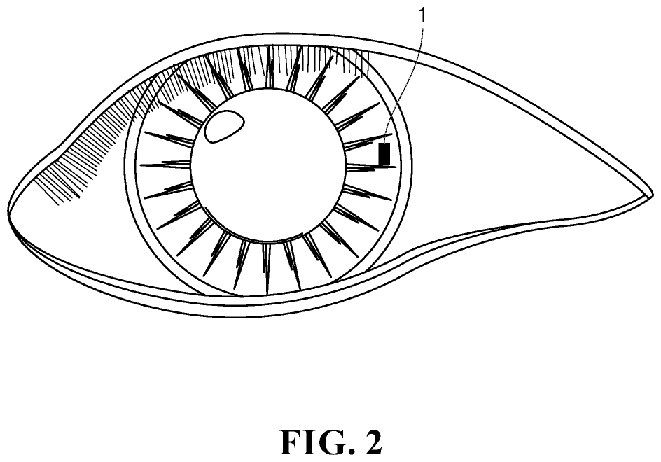

FIG. 3 shows an example optical pressure sensor in use.

FIG. 4. depicts a user and an example reader, being a handheld device.

### DETAILED DESCRIPTION

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

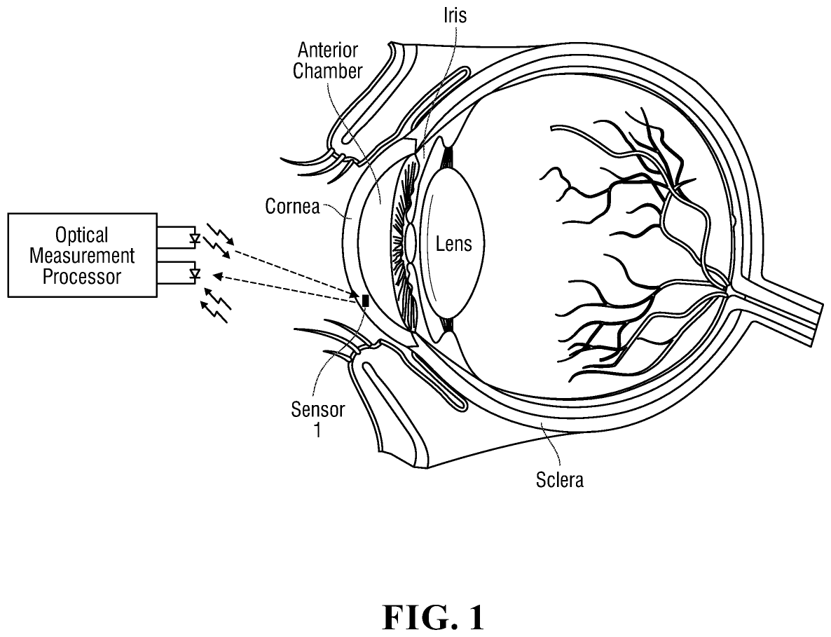

FIG. 1 shows an example of how to measure IOP using a passive optical sensor 1 that has been implanted in the cornea or in the sclera. While the drawings in this disclosure may not be to scale, they do illustrate that the sensor 1 is small enough to be implanted into a typical cornea (which is about 0.5 mm thick.) The sensor 1 is passive in that it does not have a source of stored power that is used to transmit a signal containing information about the IOP. Instead, a part of the sensor 1 is designed to bend or compress or conform according to the IOP, and that part is also designed to reflect incident optical energy. As a result, the sensor 1 changes how it reflects the optical energy, as a function of the IOP at that moment. Thus, the reflection changes in that it follows the changing IOP (in the anterior chamber), for example over the course of a day. As the sensor is passive, it can be made small and thin so as to be implanted into the cornea in a minimally invasive manner, more easily and with less risk of complications as compared to implant locations that are further inside the eye. In one aspect, the sensor is implantable in the cornea in its entirety as shown, so that it is entirely embedded in the cornea (there are no extensions or other pieces that extend beyond the cornea.) In another aspect, the sensor is implanted in a position that is closer to the limbus than the visual axis of the eye. The sensor can be made sufficiently small, e.g. having a footprint or area in the x-y plane of about 1 mm² and a thickness in the z direction of 0.2 to 0.3 mm) so that it is entirely embedded in the cornea as shown (there are no extensions or other pieces attached to the sensor that extend beyond the cornea.)

The depth of the desired incision in which the sensor is to be placed may be measured via optical coherence tomography (OCT) prior to the incision, and then also after the incision (e.g., again via OCT) to ensure that placement of the sensor is correct.

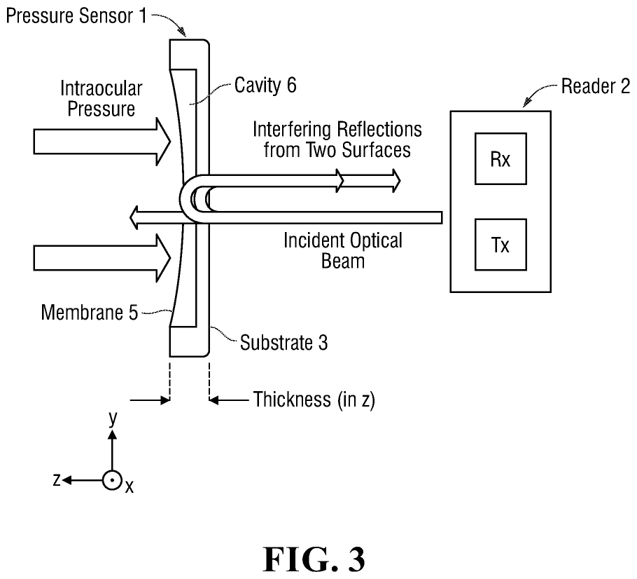

In one instance, referring now to the example shown in FIG. 3, the sensor 1 has a rigid substrate 3 to which a flexible membrane 5 (the terms rigid and flexible being relative to one another) is attached, so as to form a sealed cavity 6 that is bound by (or defined by) the internal faces of the substrate and the membrane. The membrane 5 may be spaced from the substrate in the thickness or z-direction for example between 5 to 30 microns. The membrane 5 may be made of or coated with one or more of the following biocompatible materials: silica or any material that has a suitable difference in optical index of refraction relative to that of the surrounding tissue. The substrate 3 may be made or coated with any one of similar biocompatible materials as well. The indices of refraction of the membrane and substrate surfaces should be chosen to maximize the contrast of the interference that is due to reflections from those surfaces of the sensor 1.

To make IOP measurements, a reader 2 contains an optical transmitter (Tx) and an optical receiver (Rx), both of which may be integrated within a housing of the reader. The reader is aligned with the eye so that a beam of incident optical energy (radiation or waves) emitted by the transmitter impinges on the sensor 1, while reflections of that radiation from the sensor 1 are detected by the receiver (as its output signal.) Note the term “beam” is used generically here, and does not require a beamforming transmitter array. The beam enters the cornea where it impinges (or is incident) upon the sensor 1, and is reflected by the sensor 1 towards the receiver. In the particular example of FIG. 3, the sensor 1 is oriented so that the incident beam impinges the rigid substrate 3 first, where some of the incident energy is reflected toward the receiver and some is transmitted into the cavity 6 where it is reflected (toward the receiver) off the membrane 5 as shown. An estimate of the IOP is then determined by an optical measurement processor, by digitally processing an electrical output signal of the receiver that is responsive to the interfering reflections from at least two surfaces of the sensor 1. This is possible because the output signal of the receiver changes, in a detectable manner, as the sensor 1 bends or changes shape due to the changing IOP. The system is designed such that the reader and implanted sensor don't require tight tolerances in lateral alignment (e.g., having an eyebox of at least a few hundred microns per side, where the eyebox may be defined as an area or a volume within which the transmitter, receiver and sensor should be located in order to produce reliable measurements.) This allows the user to easily align the reader to the implanted sensor with proper feedback from the reader. The system can be designed so that the user is not required to hold the reader in the aligned position for a long period of time. Instead, to make an IOP reading it is sufficient that the reader just briefly passes through the good alignment volume. For example, the reader may be taking the measurements every 1 millisecond, with the actual duration of receiver signal acquisition (sample) equal to 1 microsecond. A fast signal processing algorithm allows the measurement processor to filter out and discard any acquired digital samples if the reader and implant are not aligned relative to each other (when that sample was acquired.)

In the example of FIG. 3, the sealed cavity 6 is a gaseous volume that is made to be at a low enough pressure, for example on the order of the atmosphere (atm) or lower, that allows changes in the IOP to sufficiently bend or change the shape of the membrane 5 (via movement of the corneal tissue that surrounds the sensor 1 and that is caused by the changing IOP) so as to be detectable in the reflections. In other words, the sealed cavity has a depth that varies as a function of the IOP of the eye in which it is implanted. The gaseous volume in the sealed cavity may be air or it may be vacuum. When the sensor 1 is implanted in the orientation shown, namely that an outside surface of the membrane 5 faces the inside of the eye (or faces the anterior chamber of the eye) while the outside surface of the substrate 3 faces the transmitter Tx (or faces the outside environment of the eye), the incident beam will be reflected at least four times including at a boundary between tissue and substrate, a boundary between substrate and cavity, a boundary between cavity and membrane and finally a boundary between the membrane and tissue. Typically, the two reflections at the cavity boundaries are what are most important and the other two reflections may be minimized with antireflection coatings or with appropriate choice of material. More generally, the membrane 5 changes shape as a function of IOP, and so changes the distance between two reflection boundaries.

The processor analyzes the signal from the receiver Rx to interpret the reflections from the sensor 1 into an estimate of the IOP, e.g., in units of mmHg. The processor may look for a spectral frequency dependent reflectivity characteristic in the receiver output signal, which can be correlated to how much the sensor 1 is being bent or compressed (by the IOP.) As such the processor may operate as a spectrometer (that performs a spectroscopy algorithm.) In another aspect, the processor analyzes the signal from the receiver Rx in a spatial sense, to determine or evaluate an interference pattern that is produced by the reflections (where the interference pattern changes as a function of bending of the membrane.) The processor may determine an absolute pressure reading as the pressure that is exerted on the sensor. To determine the pressure in the eye relative to the ambient pressure (which is typically what is needed for IOP), an ambient pressure sensor could be used in the reader and this reading can be subtracted from the absolute pressure reading.

Note that some or all of the digital signal processing that is performed upon the receiver output signal (by the optical measurement process) may be performed by a digital processor which is inside the reader 2. That digital processor may alternatively be inside a companion device such as a smartphone which is wirelessly paired for data communication with the reader 2, and the reader 2 transmits a digital version of the receiver output signal to the companion device for processing. Some or all of the digital processing may be relegated to a cloud computing service.

A wavelength range of the transmitted optical beam may be selected so as to increase the signal to ratio at the receiver output (which is detecting reflections from the implanted sensor 1.) In one aspect, the optical beam is within the wavelength range 750 nm to 1080 nm, such that the user cannot perceive the optical beam. In one instance, the selected wavelength may be one that results in low scattering (by skin and by the corneal tissue that surrounds the implanted sensor 1) over the first 100 microns of depth but then high scattering at greater depths (beyond the depth at which the sensor 1 is implanted.)

In one aspect, the transmitter is controlled so as to emit a variety of optical frequencies. The transmitter may be configured to produce a linear chirp or a frequency sweep or other time dependent (time varying) optical waveform, acting to interrogate the sensor 1. In another aspect, the transmitter is configured to produce the interrogating, optical beam as a noise-like waveform. In yet another aspect, the transmitter is configured to produce the interrogating optical beam as a narrow band signal or single color that is coherent (has a stable and controlled phase), and the measurement processor is configured to perform image analysis of the spatial reflection pattern in the receiver output signal. To improve signal to noise ratio (and reduce interference from other optical sources), the transmitted beam could be modulated with a code, which would be detectable when processing the output signal of the receiver. The transmitted beam could also be modulated to increase eye safety (i.e., the detector is only active when the transmitted beam is pulsed).

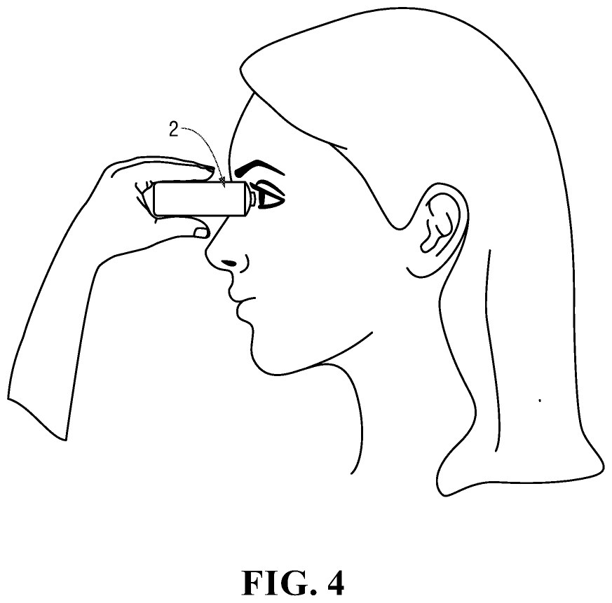

The reader 2 may be a handheld device as illustrated in FIG. 4, for example a consumer focused product that is to be held in the hand of the person, while being aimed at the front of person's eye (in which the sensor 1 is implanted.) Alternatively, the reader 2 could be placed on the head of the person, or affixed to a bench or stand next to the person. The person may be instructed to look towards the reader or straight ahead, and keep a fixed gaze directly forward which may facilitate alignment of the sensor 1 with the transmitter-receiver pair. In one aspect, the sensor 1 has a physical registration feature that is detectable using an optical imaging function in the reader 2, where the processor uses the detected registration feature to verify that the reader 2 is aligned with the sensor 1, e.g., estimate a position and/or orientation of the sensor 1. The registration feature may also encode a serial number of the sensor 1 (that is also detectable by the imaging function of the reader 2.)

The reader 2 may have an optical lens that focuses the incident optical beam being emitted by the transmitter. The transmitter may be a single photo-emitter, or it may be an array of light emitters. The emitted beam may be directional, having a narrow or directional primary lobe aimed at the sensor 1. The receiver may be a single photo-detector, or it may be a one-dimensional or two-dimensional array of photo-detectors (the latter being especially useful for the case where the processor is determining the interference pattern via for example an imaging function being performed upon the signals produced by the array of photo-detectors.) A focused or narrowed incident beam may be combined with a scanning mechanism, either mechanical or, in the case of a beamforming array, a scanning array algorithm, that can be used to sweep an area where the sensor 1 is expected to be located so as to reduce the constraints on how the reader is to be positioned in relation to the sensor 1.

Since the cornea is not actually inside the eye (unlike the anterior chamber which is filled with the aqueous humor), an IOP estimate that is determined using a pressure sensor implanted in the cornea is not as direct a measurement of the IOP as would be obtained using a sensor that is for example within the anterior chamber of the eye. As such, one or more parameters may need to be determined for example by a calibration procedure that is performed with the reader and the sensor as implanted. The parameter may account for the indirectness of the measurement. The parameter relates changes in IOP to corresponding changes in the cornea that cause the implanted sensor to bend or compress, during reflection of the incident beam that is detected by the receiver. The parameter's value may be different for each instance of the implanted sensor (in different eyes of the same person and in different persons) as it may also be a function of for example the depth (in the thickness direction) of the implant location in the cornea, or more generally the position and/or orientation of the implanted sensor. Such a parameter may be for instance a scaling factor and/or an additive offset that is applied by a digital processor to a reading of the output signal of the receiver. In the case where the parameter is a scaling factor, that value will be closer to unity the deeper the sensor is implanted (closer to the anterior chamber.) The parameter may alternatively be part of a more complex set of parameters that are applied to the receiver readings, for example by a machine learning model. In most instances, since the placement of the sensor 1 will not shift much after surgical implantation, the parameter can be calibrated once the eye has healed from the surgery.

While certain aspects have been described and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. For example, while FIG. 1 illustrates the transmitter and receiver as photodiode symbols, other types of photo-emitters and photo-detectors may be used in the transmitter and the receiver. The description is thus to be regarded as illustrative instead of limiting.

What is claimed is:

1. An intraocular pressure (IOP) measurement system comprising:
an optical pressure sensor implantable in the cornea of an eye, wherein the sensor has a sealed cavity and a membrane that changes shape as a function of IOP of the eye;
an optical transmitter to emit an incident optical beam;
a receiver to produce an output signal in response to receiving a plurality of reflections of the incident optical beam from the sensor; and
a processor configured to estimate the IOP of the eye based on processing the output signal of the receiver.

2. The system of claim 1 wherein the sensor is implantable in the cornea in its entirety so that it is entirely embedded in the cornea.

3. The system of claim 1 wherein the sensor provides a frequency dependent reflection of the incident optical beam, that changes as a function of the TIP.

4. The system of claim 3 wherein the sensor comprises a rigid substrate and a flexible membrane attached to the substrate that define the sealed cavity.

5. The system of claim 4 wherein the sealed cavity is a gaseous cavity.

6. The system of claim 4 wherein an outside surface of the rigid substrate faces the transmitter and an outside surface of the membrane faces the inside of the eye.

7. The system of claim 1 wherein the processor determines, by processing the output signal of the receiver, an optical interference pattern which varies as a function of the IOP.

8. The system of claim 1 wherein the transmitter and the receiver are integrated within a single housing of a reader.

9. The system of claim 8 wherein the reader is a handheld device, and the processor is outside of the handheld device.

10. The system of claim 1 wherein the sensor is implanted in a position that is closer to the limbus than the visual axis of the eye.

11. A method for measuring IOP of an eye, the method comprising:
emitting an optical beam toward the eye;
detecting, as an output signal, reflections of the optical beam from a pressure sensor that is implanted in a cornea of the eye; and
processing the output signal to compute an estimate of the IOP of the eye.

12. The method of claim 11 wherein processing the output signal comprises performing a spectroscopy algorithm.

13. The method of claim 12 wherein the sensor is implanted in the cornea in its entirety so that it is entirely embedded in the cornea.

14. The method of claim 11 wherein processing the output signal comprises computing an estimate of frequency dependent impedance presented to the incident optical beam, that changes as a function of the IOP.

15. The method of claim 11 wherein the sensor comprises a rigid substrate and a flexible membrane attached to the substrate that define the sealed cavity.

16. The method of claim 15 wherein the sealed cavity is a gaseous cavity.

17. The method of claim 15 wherein the substrate is transparent to and the membrane is reflective of the incident optical beam.

18. The method of claim 11 wherein emitting, detecting and processing are performed by electronics that are integrated within a single housing of a reader.

19. The method of claim 11 emitting and detecting are performed by electronics that are integrated within a single housing of a reader, wherein the reader is a handheld device, and the method further comprises transmitting a digital version of the output signal to a digital processor that is outside of the handheld device and that performs the processing of the output signal to compute the estimate of the IOP of the eye.
